(19) United States
(12) Patent Application Publication
(10) Pub. No.: US 2005/0148948 A1
(43) Pub. Date: Jul. 7, 2005

(54) Sutureless ophthalmic drug delivery system and method

(76) Inventor: Steven Gerard Caputa, St. Louis, MO (US)

Correspondence Address: Michael L. Smith;Bausch & Lomb Incorporated, One Bausch & Lomb Place, Rochester, NY, 14604-2701, US

(21) Appl. No.: 10/741,754

(22) Filed: Dec. 19, 2003

(51) Int. Cl. A61M 5/00

(52) U.S. Cl. 604/264

(57) ABSTRACT

A sutureless ophthalmic drug delivery system includes a transconjunctival entry alignment device 10 for insertion into a patient's eye 54 and at least one drug delivery device 34 capable of being inserted into the eye 54 through the entry alignment device 10.

### BACKGROUND OF THE INVENTION

1. Field of the Invention

The present invention is directed to a system for inserting a drug delivery device into an eye suturelessly.

2. Description of Related Art

Surgery in general and ophthalmic surgery in particular, is constantly developing less and less invasive surgical techniques in order to minimize the trauma inflicted upon the patient during surgery and minimize the recovery time required to overcome the trauma of surgery.

To this end, sutureless surgery techniques for ophthalmic surgery have been developed, such as the TSV25™ System available from Bausch & Lomb Incorporated. The TSV25™ System has allowed surgeons to perform surgery more quickly and efficiently by streamlining the opening and closing procedures of surgery. Because the TSV25™ System inserts an entry alignment cannula without prior incisions transconjunctivally through the eye no resection of the conjunctiva is required. Also, after surgery is completed the entry alignment device or cannula is simply removed from the eye, and because of the small size of the cannula the wounds through the eye are self-healing and do not require uncomfortable and irritating sutures.

Also, being developed are controlled drug delivery devices that release a controlled amount of pharmaceutical through a permeable outer barrier over a period time to treat a variety of retinal diseases, such as macular degeneration and other diseases of the eye. Procedures for inserting such drug delivery devices into the eye heretofore required relatively large incisions to be made in the eye and sutures to be used to close the wounds after the operation has been completed.

Therefore, it would be highly desirable to provide a sutureless drug delivery system for decreasing the surgery time required, increasing the efficiency of the procedure, speeding the recovery time, and raising the satisfaction of the patient with the procedure.

### BRIEF DESCRIPTION OF THE DRAWINGS

FIG. 1 is an exploded elevation of a trocar and an entry alignment device, which form a portion of the present invention;

FIG. 2 is a perspective view of an entry alignment device of FIG. 1;

FIG. 3 shows a light pipe, which forms part of a kit in accordance with the present invention;

FIG. 4 shows a forceps, which forms a part of a kit in accordance with the present invention;

FIG. 5 shows an irrigation line, which forms a part of a kit in accordance with the present invention;

FIG. 6 shows a suture, which forms a part of a kit in accordance with the present invention;

FIG. 7 shows a container of adhesive, which forms a part of a kit in accordance with the present invention;

FIG. 8 shows a laser endoprobe, which forms a part of a kit in accordance with the present invention;

FIG. 9 shows an aspirating pick, which forms a part of a kit in accordance with the present invention;

FIG. 10 shows a vitrectomy cutter tip, which forms a part of a kit in accordance with the present invention;

FIG. 11 shows a drug delivery device, which forms a part of a kit in accordance with the present invention;

FIG. 12 shows an exploded view of one embodiment of a kit in accordance with the present invention; and

FIG. 13 shows a surgical procedure in accordance with the present invention.

### DETAILED DESCRIPTION

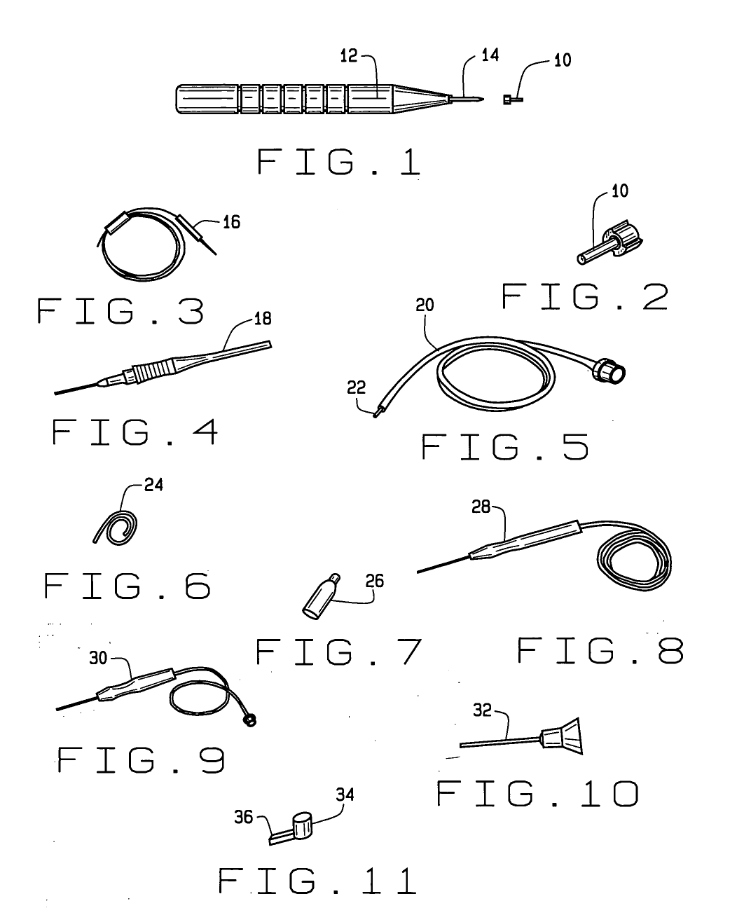

FIG. 1 shows an exploded view of a transconjunctival entry alignment device 10 and trocar 12. Entry alignment device 10 is for insertion into a patient's eye and is inserted into a patient's eye by being placed over trocar 12 and pointed tip 14. Such a system is well known, such as the TSV25™ System available from Bausch & Lomb Incorporated. While the present invention is described with regard to the use of a trocar 12, it would also be possible that entry alignment device 10 has a sharp pointed tip incorporated within the device itself, to eliminate the need for a trocar 12. In addition, it is also possible that entry alignment device 10 be fitted over a suitably sized needle and inserted through the eye transconjunctivally. The transconjunctival nature of the entry alignment device 10 is what allows for efficient and timely surgery for the present inventive drug delivery system. The use of a transconjunctival entry alignment device 10 in one or more places, as described further in detail below, allows for a surtureless surgery to be accomplished.

Typically in surgery, multiple entry alignment devices 10 will be used so that a variety of instruments and devices may be inserted into the eye without the need for sutures to close the wounds after surgery is completed. Such devices include a light pipe 16 of FIG. 3, a forceps 18 of FIG. 4, and an irrigation line 20, including a tip 22 that fits within entry alignment device 10 shown in FIG. 5. FIGS. 6 and 7 shwo attachment materials for use with attachment instruments. FIG. 6 shows suture 24, which may be used to attach at least one drug delivery device shown below to a patient's eye, and FIG. 7 shows a container of adhesive 26, which may be used to attach a drug delivery device in accordance with the present invention to a patient's eye. FIG. 8 shows a laser endoprobe, which may form a part of a kit, in accordance with an embodiment of the present invention. FIG. 9 shows an aspirating pick 30, of course aspirating pick 30 may also be extendible (not shown). FIG. 10 shows a vitrectomy cutting tip 32 for use with a vitrectomy cutting instrument, such as the Lightning™ available from Bausch & Lomb Incorporated. Of course, instead of a vitrectomy tip 32 a kit could also include an entire vitrectomy cutting tool and aspiration line if a disposable vitrectomy instrument is to be used. FIG. 11 shows a drug delivery device 34, in accordance with the present invention, and preferably includes an attachment tab 36 for attaching the device 34 to a patient's eye.

Drug delivery device 34 is preferably of a type made from a permeable membrane which holds a pharmaceutical to be released over an extended period of time for treating various diseases of the eye, such as macular degeneration. Such devices are described in U.S. Pat. Nos. 6,548,078 and 6,375,972 and also include the Envision™ devices currently in clinical testing by Bausch & Lomb Incorporated.

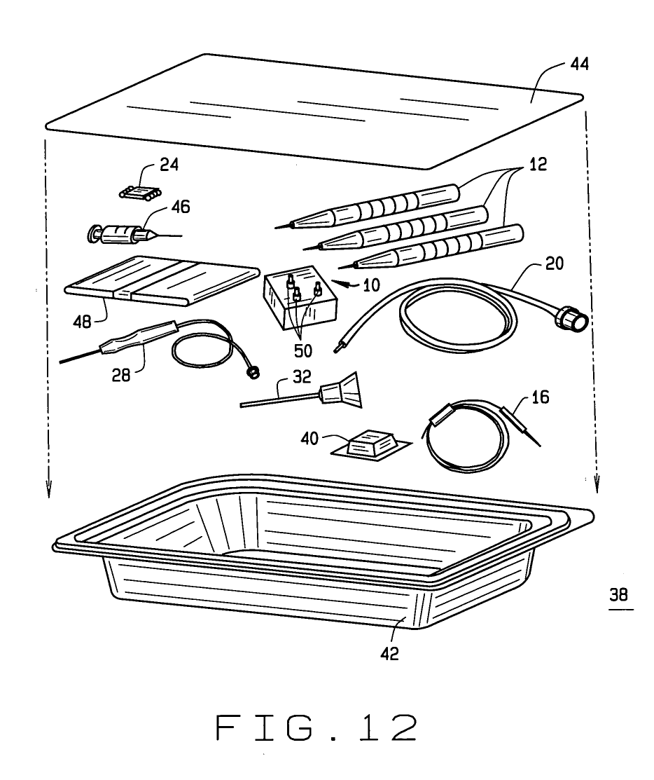

It is important that device 34 fit within entry alignment device 10 so that drug delivery device 34 can be inserted into the eye without the need for sutures. Preferably, drug delivery device 34 will have to be less than 25 gauge in diameter in order to fit within an inner lumen of entry alignment device 10. Each of the FIGS. 1 through 11 are examples of instruments that may be used and contained within a kit for ophthalmic surgery and are available from Bausch & Lomb Incorporated. Depending on the type of drug to be delivered and the disease state of the eye, the drug delivery device 34 may or may not need to be attached to a portion of the eye; therefore, at a minimum, a sutureless ophthalmic drug delivery kit 38 should include at least one transconjunctival entry alignment device 10 for insertion into a patient's eye and at least one drug delivery device 34 capable of being inserted into the eye through the entry alignment device 10, (shown in FIG. 12 as package 40 in which drug delivery device 34 is contained). Preferably, each of the entry alignment device 10 and the drug delivery device 34 are contained in a single package or kit 38. Package 38 preferably includes a tray 42 and a lid or top 44. After insertion of the kit materials and devices lid 44 will be sealed to tray 42 in a sterile fashion well known in the art. Package 38 may also include one or more trocars 12, light pipe 16, irrigation line 20, sutures 24, adhesive within syringe 46, a laser probe 28, a vitrectomy tip 32, drape 48, or plugs 50 for insertion into entry alignment device 10. These and other instruments may be included in the kit depending on the type of surgery the kit is assembled for. The pieces shown in FIG. 12 are for illustration purposes only and should not be seen as limiting.

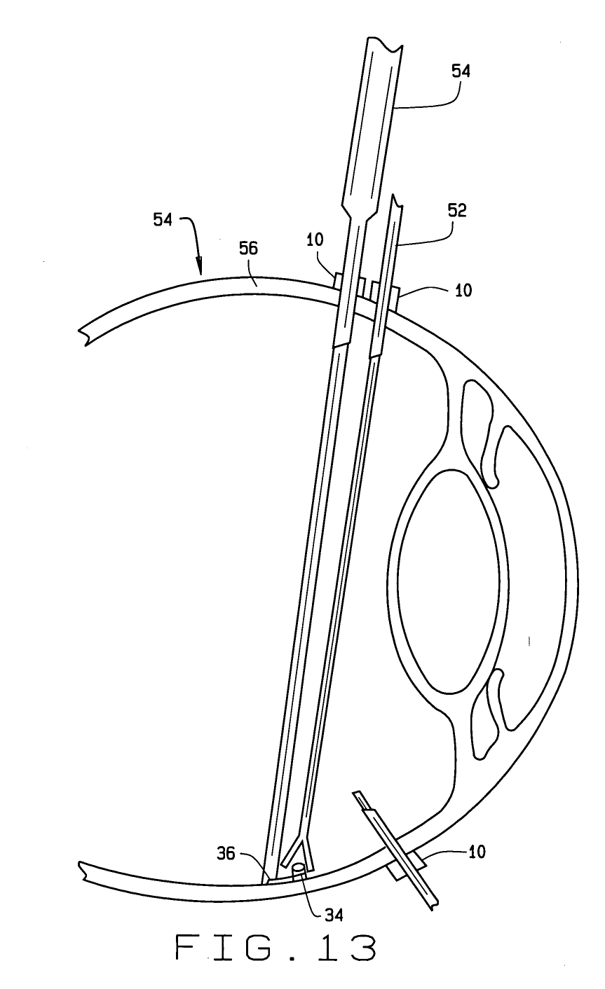

The package 38 may also further include an insertion tool 52, such as shown in FIG. 13, for inserting the drug delivery device through the entry alignment device 10 and into the eye 54. For purposes of FIG. 13, the portions of the eye through which entry alignment devices 10 are inserted and shown at 56 should be taken to include the conjunctiva and other layers of the eye through which the entry alignment devices 10 pass. Insertion tool 52 may be a forceps, as shown, or could be a specially designed instrument for holding drug delivery device 34 while passing through entry alignment device 10 and eye 54. Depending on the application and type of surgery to be conducted, an attachment instrument 54 may be used to attach drug delivery device 34 to an eye, preferably via a tab 36. Attachment instrument 54 may be a bipolar handpiece for thermally attaching tab 36 to eye 54 or it could be other instruments, such as a syringe containing adhesive or a needle for use with sutures to attach tab 36 to eye 54 or some other known attachment instrument.

A typical surgery will include the steps of inserting an entry alignment device 10 transconjunctivally into the eye 54 and passing at least one drug delivery device 34 through the entry alignment device 10 and into the eye 54. The surgery may also include the use of insertion tool 52 for inserting the drug delivery device 34 through the entry alignment device 10. Furthermore, the surgery may also include attaching a drug delivery device 34 to a portion of the eye 54 with an attachment instrument 54. If drug delivery device 34 is to be attached to eye 54, then preferably the surgery includes inserting a vitrectomy instrument, such as an instrument using tip 32 through the entry alignment device 10 for performing a vitrectomy procedure prior to the insertion of the drug delivery device 34 into the eye 54.

1. A sutureless ophthalmic drug delivery system comprising:
at least one transconjunctival entry alignment device for insertion into a patient's eye; and
at least one drug delivery device capable of being inserted into the eye through the entry alignment device.

2. The system of claim 1 further including an attachment instrument capable of being inserted through the entry alignment device and for attaching the drug delivery device to a portion of the eye.

3. The system of claim 2 further including attachment material for use with the attachment instrument.

4. The system of claim 3, wherein the attachment material includes sutures.

5. The system of claim 3, wherein the attachment material includes adhesive.

6. The system of claim 1 further including an insertion tool for inserting the drug delivery device through the entry alignment device and into the eye.

7. A sutureless ophthalmic drug delivery surgical kit comprising:
at least one transconjunctival entry alignment device for insertion into a patient's eye;
at least one drug delivery device capable of being inserting into the eye through the entry alignment device; and
wherein each of the entry alignment device and the drug delivery device are contained in a single package.

8. The kit of claim 7, wherein the package further includes an insertion tool for inserting the drug delivery device through the entry alignment device and into the eye.

9. The kit of claim 7, wherein the package further includes an attachment instrument capable of being inserted through the entry alignment device and for attachment of the drug delivery device to a portion of the eye.

10. The kit of claim 7, wherein the package further includes at least a portion of a vitrectomy instrument capable of being inserted through the entry alignment device and for performing a vitrectomy procedure prior to insertion of the drug delivery device into the eye.

11. The kit of claim 7, wherein the package further includes one or more of a trocar for inserting the entry alignment device into the eye, a viscoelastic, a silicone oil, a drape, a light pipe, a forceps, a plug for connection to the entry alignment device, a suture, an adhesive, a laser probe, a bipolar handpiece, and an aspirating pick.

12. A sutureless ophthalmic drug delivery surgical kit comprising:
at least one transconjunctival entry alignment device for insertion into a patient's eye;
at least one drug delivery device capable of being inserting into the eye through the entry alignment device;
wherein each of the entry alignment device and the drug delivery device are contained in a single package;
wherein the package further includes an insertion tool for inserting the drug delivery device through the entry alignment device and into the eye;
wherein the package further includes an attachment instrument capable of being inserted through the entry alignment device and for attachment the drug delivery device to a portion of the eye; and
wherein the package further includes at least a portion of a vitrectomy instrument capable of being inserted through the entry alignment device and for performing a vitrectomy procedure prior to insertion of the drug delivery device into the eye.

13. The kit of claim 12, wherein the package further includes one or more of a trocar for inserting the entry alignment device into the eye, a viscoelastic, a silicone oil, a drape, a light pipe, a forceps, a plug for connection to the entry alignment device, a suture, an adhesive, a laser probe, a bipolar handpiece, and an aspirating pick.

14. A sutureless method of inserting a drug delivery device into a patient's eye comprising the steps of:
inserting at least one entry alignment device transconjunctivally into the eye; and
passing at least one drug delivery device through the entry alignment device and into the eye.

15. The method of claim 14 further including the step of providing an insertion tool for inserting the drug delivery device through the entry alignment device.

16. The method of claim 14 further including the step of attaching the drug delivery device to a portion of the eye.

17. The method of claim 14 further including the step of inserting a vitrectomy instrument through the entry alignment device for performing a vitectomy procedure prior to insertion of the drug delivery device into the eye.
